(19) United States
(12) Patent Application Publication
(10) Pub. No.: US 2024/0399006 A1
(43) Pub. Date: Dec. 5, 2024

(54) APPARATUS AND A PROCESS FOR STERILIZING AND RINSING CONTAINERS

(71) Applicant: GEA PROCOMAC S.P.A., SALA BAGANZA (IT)

(72) Inventors: ANDREA COMANI, SALA BAGANZA (IT); ANGELO FORNARI, COLORNO (IT); BARBARA BRICOLI, MEDESANO (IT)

(21) Appl. No.: 18/675,600

(22) Filed: May 28, 2024

(30) Foreign Application Priority Data
May 30, 2023 (IT) 102023000010905

(51) Int. Cl. A61L 2/26; A61L 2/18; A61L 101/34; B08B 9/20; B08B 9/32; B08B 9/34

(52) U.S. Cl. CPC A61L 2/26; A61L 2/186; B08B 9/205; B08B 9/32; B08B 9/34; A61L 2101/34; A61L 2202/17; A61L 2202/23; B08B 2209/08

(57) ABSTRACT

A process for sterilizing and rinsing containers, the process comprising the following steps: sterilizing the containers with a sterilizing solution coming from a first tank; after sterilization, subjecting the containers to a first rinsing by dispensing towards them a first amount of sterile water from a UHT device in a first rinsing tract of an advancement path of the containers; after the first rinsing, subjecting the containers to a second rinsing by dispensing towards them a second amount of sterile water from said UHT device in a second rinsing tract of the path; and collecting at least part of exhaust sterile water dropping from the containers undergoing the second rinsing and sending it to the UHT device.

The present invention relates to an apparatus and a process for sterilizing and rinsing containers.

In an aseptic bottling line, there is the need to sterilize the containers formed before they are filled.

In this context, the attention is focused on chemical sterilization, in which the external and internal surfaces of the containers are treated with hydrogen peroxide, acetic acid or peracetic acid.

After the chemical treatment, the containers are rinsed with sterile water.

The main issue of chemical sterilization is connected with the disposal of chemical residues and the consumption of water for the rinsing step.

In accordance with a known solution, the rinsing water is purified by an active carbon filter, and it is then partially recycled in the process.

In another solution, the rinsing water is passed through a catalyst and then in an ion exchange resin.

In this context, the object of the present invention is to provide an apparatus and a process for sterilizing and rinsing containers, which overcome the problems of the prior art cited above.

In particular, the object of the present invention is to propose an apparatus and a process for sterilizing and rinsing containers, which reduce the water consumption for the rinsing step.

Another object of the present invention is to propose an apparatus and a process for sterilizing and rinsing containers, having a lower environmental impact with respect to the known solutions.

The stated technical task and specified aims are substantially achieved by an apparatus for sterilizing and rinsing containers, comprising:
a sterilizing unit configured to treat the containers by means of a sterilizing solution contained in a first tank;
a rinsing unit arranged downstream the sterilizing unit and configured to rinse the containers advancing along a path with sterile water;
a UHT device configured to provide sterile water to the rinsing unit;
dispensing means in selective fluid communication with the UHT device so as to dispense a first amount of sterile water towards the containers in a first rinsing tract of the path and a second amount of sterile water towards the containers in a second rinsing tract of the path that is downstream the first rinsing tract;
a recovery collector arranged at the second rinsing tract of the path for collecting at least part of exhaust sterile water dropping from the containers advancing along the second rinsing tract, said recovery collector being in selective fluid communication with the UHT device.

According to one embodiment of the invention, the dispensing means are configured to dispense sterile water by a single dispensing pulse which supplies both the first amount of sterile water and the second amount of sterile water.

According to one embodiment of the invention, the apparatus comprises a further recovery collector arranged at the first rinsing tract of the path for collecting at least part of the exhaust sterile water dropping from the containers advancing along the first rinsing tract.

In particular, the further recovery collector is in selective fluid communication with the first tank.

According to an aspect of the invention, the recovery collector and the further recovery collector are separated.

In a preferred embodiment of the invention, the apparatus comprises two distinct piping systems configured to establish a selective fluid communication respectively between the recovery collector and the UHT device and between the further recovery collector and the first tank.

According to one embodiment of the invention, the sterilizing unit and the rinsing unit comprise a corresponding rotating carousel.

In an alternative embodiment of the invention, the sterilizing unit and the rinsing unit comprise a corresponding linear conveyor or belt.

In a preferred embodiment of the invention, the apparatus further comprises a second tank arranged downstream the UHT device so as to receive the sterile water from it. In particular, the second tank is configured to supply the sterile water to the dispensing means. The stated technical task and specified aims are substantially achieved by
a process for sterilizing and rinsing containers, the process comprising the following steps:
   sterilizing the containers with a sterilizing solution coming from a first tank;
   after sterilization, subjecting the containers to a first rinsing by dispensing towards them a first amount of sterile water from a UHT device in a first rinsing tract of an advancement path of the containers;
   after the first rinsing, subjecting the containers to a second rinsing by dispensing towards them a second amount of sterile water from said UHT device in a second rinsing tract of the path;
   collecting at least part of exhaust sterile water dropping from the containers undergoing the second rinsing and sending it to the UHT device.

In particular, the exhaust sterile water which is collected and sent to the UHT device is used in rinsing new containers advancing along the path.

According to one embodiment of the process, the first rinsing step and the second rinsing step are performed by dispensing means which are configured to dispense sterile water by a single dispensing pulse which spans both the first rinsing step and the second rinsing step.

In particular, the dispensing means are configured to dispense the first amount of sterile water and the second amount of sterile water without any interruption.

According to one embodiment of the invention, the sterile water obtained in the UHT device is sent to a second tank which in turn is configured to dispense the sterile water towards the containers during the first rising and the second rinsing.

In a preferred embodiment, the process further comprises a step of collecting at least part of exhaust sterile water dropping from the containers undergoing the first rinsing and sending it to the first tank.

In particular, the exhaust sterile water collected in the first rinsing tract is mixed with the sterilizing solution of the first tank for being used in sterilizing new containers.

Further characteristics and advantages of the present invention will more fully emerge from the non-limiting description of a preferred but not exclusive embodiment of an apparatus and a process for sterilizing and rinsing containers, as illustrated in the accompanying drawings in which:

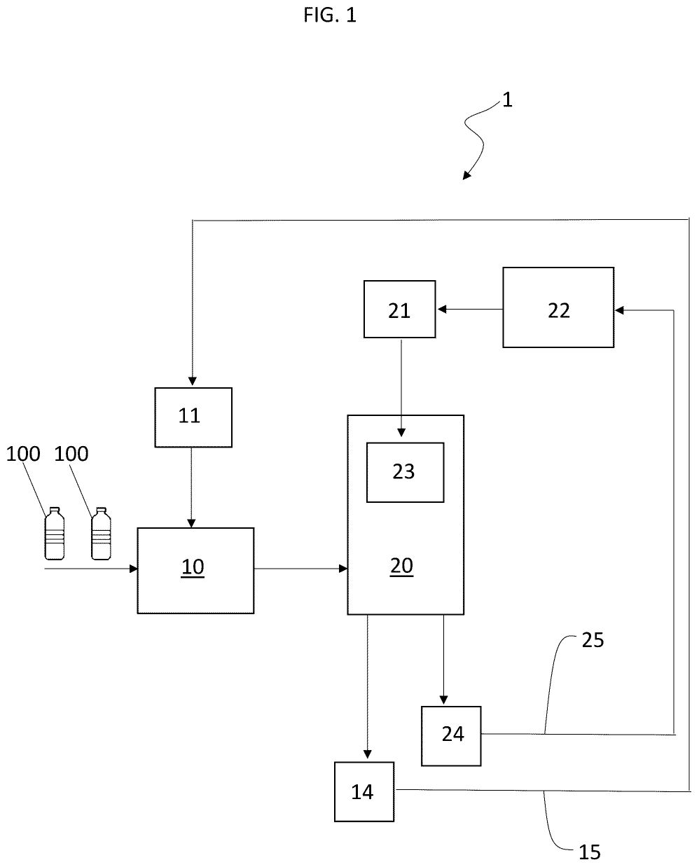

FIG. 1 schematically illustrates an apparatus for sterilizing and rinsing containers, according to the present invention;

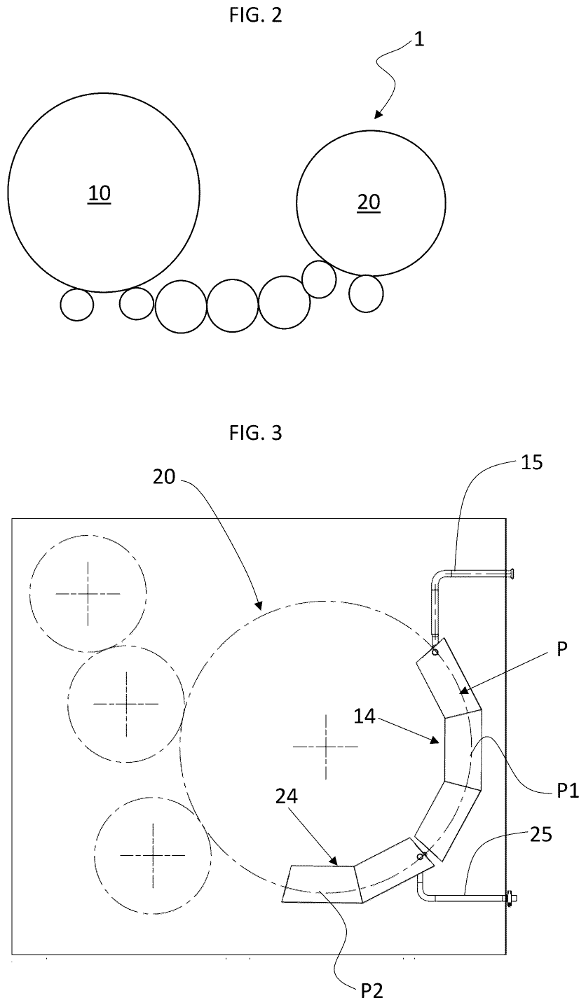

FIG. 2 is a plain view of an embodiment of an apparatus for sterilizing and rinsing containers, according to the present invention;

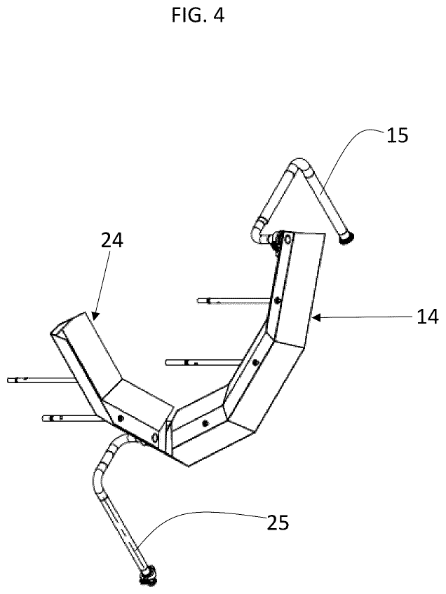

FIG. 3 illustrates the simplified structure of a part of the apparatus of FIG. 2 (focusing on the rinsing unit), in plain view;

FIG. 4 illustrates the collectors arranged at the rinsing unit of FIG. 3, in a perspective view.

With reference to the drawings, number 1 denotes an apparatus for sterilizing and rinsing containers 100, in particular made of a thermoplastic material.

The apparatus 1 comprises a sterilizing unit 10 and a rinsing unit 20 arranged downstream the sterilizing unit 10.

The sterilizing unit 10 is configured to treat the containers 100 by means of a sterilizing solution.

In particular, the apparatus 1 comprises a first tank 11 containing a sterilizing solution, which is a chemical compound based on one or more species of peroxides and/or organic acids.

In one example, the first tank 11 contains a peracetic acid solution.

The rinsing unit 20 is configured to rinse the containers 100 using sterile water.

In particular, the apparatus 1 comprises a UHT device 22 configured to provide sterile water, obtained by thermal treatment, to the rinsing unit 20.

Preferably, the apparatus 1 comprises a second tank 21 arranged downstream the UHT device 22 so as to receive the sterile water from it.

In practice, the sterile water obtained in the UHT device 22 is supplied to the second tank 21.

In the rinsing unit 20 the containers 100 are made to advance along a path P.

The apparatus 1 further comprises dispensing means 23 in selective fluid communication with the UHT device 22.

In one embodiment of the invention, illustrated in FIG. 1, the second tank 21 is configured to receive the sterile water from the UHT device 22 and to supply it to the dispensing means 23.

The dispensing means 23 are configured to dispense a first amount of sterile water towards the containers 100 in a first rinsing tract P1 of the path P and a second amount of sterile water towards the containers 100 in a second rinsing tract P2 of the path P.

In accordance with an embodiment of the invention, the dispensing means 23 are part of the rinsing unit 20.

Preferably, the dispensing means 23 comprise a plurality of nozzles.

More preferably, the nozzles are external to the containers 100 and are configured to dispense sterile water on internal walls of the containers 100.

In an alternative embodiment, the nozzles are also configured to penetrate inside the containers 100 so as to dispense sterile water on internal walls of the containers 100.

When the containers 100 move in the rinsing unit 20 they advance along the first rinsing tract P1 and then along the second rinsing tract P2.

The second rinsing tract P2 is downstream the first rinsing tract P1 with respect to the advancement direction of the containers 100 along the path P in the rinsing unit 20.

In one example, the second rinsing tract P2 is immediately consecutive to the first rinsing tract P1. In another example, the second rinsing tract P2 is successive to the first rinsing tract P1 but separated from that.

Preferably, the dispensing means 23 are configured to dispense sterile water by a single dispensing pulse which supplies both the first amount of sterile water and the second amount of sterile water.

Alternatively, the dispensing means 23 are configured to dispense the first amount of sterile water with a first dispensing pulse, and the second amount of sterile water with a second dispensing pulse. In practice, the second dispensing pulse occurs after the first dispensing pulse has finished.

Advantageously, the apparatus 1 further comprises a recovery collector 24 arranged in the rinsing unit 20, in particular at the second rinsing tract P2 for collecting at least one part of the exhaust sterile water dropping from the containers 100 advancing along the second rinsing tract P2.

The recovery collector 24 is in selective fluid communication with the second tank 21 via the UHT device 22 so as to recover the collected exhaust sterile water dropping from the containers 100 in the second rinsing tract P2.

Preferably, the apparatus 1 comprises a further recovery collector 14 arranged in the rinsing unit 20, in particular at the first rinsing tract P1 for collecting at least part of the exhaust sterile water dropping from the containers 100 advancing along the first rinsing tract P1.

The further recovery collector 14 is in selective fluid communication with the first tank 11 so as to recover the collected exhaust sterile water dropping from the containers 100 in the first rinsing tract P1.

In particular, the recovery collector 24 and the further recovery collector 14 are separated, i.e. they are not in fluid communication. As a matter of fact, they are configured to collect exhaust sterile water in two different zones of the rinsing unit 20, which correspond to different contents of peroxides.

Preferably, the apparatus 1 comprises a piping system 25 configured to establish a selective fluid communication between the recovery collector 24 and the UHT device 22, which in turn feeds the second tank 21. In particular, the piping system 25 is configured to channelize the exhaust sterile water recovered from the recovery collector 24 towards the UHT device 22.

For example, the piping system 25 comprises a pump.

Analogously, the apparatus 1 comprises a further piping system 15 configured to establish a selective fluid communication between the further recovery collector 14 and the first tank 11. In particular, the further piping system 15 is configured to channelize the exhaust sterile water recovered from the further recovery collector 14 towards the first tank 11.

For example, the further piping system 15 comprises another pump.

Preferably, the recovery collector 24 and the further recovery collector 14 are each designed as leak pans configured to collect dropping water.

Said leak pans are respectively arranged below the first rinsing tract P1 and the second rinsing tract P2, as shown in FIG. 3.

According to one embodiment of the invention, illustrated in FIG. 2, the apparatus 1 is of the rotating type. Therefore, both the sterilizing unit 10 and the rinsing unit 20 comprise a rotating carousel.

In this embodiment, the path P along the rinsing unit 20 is a curvilinear path. The first rinsing tract P1 and the second rinsing tract P2 are arcuate portions of the curvilinear path P.

According to another embodiment of the invention (not illustrated), the apparatus 1 is of the linear type. Therefore, both the sterilizing unit 10 and the rinsing unit 20 comprise a linear conveyor or belt.

In this embodiment, the path P along the rinsing unit 20 is a linear path.

The first rinsing tract P1 and the second rinsing tract P2 are linear portions of the linear path P.

The process for sterilizing and rinsing containers, according to the present invention, is described hereafter.

The containers 100 are first sterilized with a sterilizing solution.

In the embodiment described and illustrated herewith, the containers 100 are sterilized in the sterilizing unit 10 by the sterilizing solution coming from the first tank 11.

After having been sterilized, the containers 100 are rinsed. According to one aspect of the invention, the containers 100 undergo two rinsing steps.

In particular, both the rinsing steps occur in the rinsing unit 20.

In a first rinsing tract P1 of the path P of the containers 100 in the rinsing unit 20, the containers 100 undergo a first rinsing step. For the first rinsing step it is employed a first amount of sterile water coming from the second tank 21.

The first amount of sterile water is dispensed towards the containers 100 when they are moving along the first rinsing tract P1.

In particular, the first amount of sterile water is dispensed by nozzles (not illustrated) which are in selective fluid communication with the UHT device 22 (and in particular with the second tank 21).

At least a part of exhaust sterile water used during the first rinsing step is recovered for being used for sterilizing new containers 100.

In particular, part of the exhaust sterile water dropping from the containers 100 which undergo the first rinsing step is collected by the further recovery collector 14 and sent back to the first tank 11.

This part of exhaust sterile water recovered from the first rinsing step is called here “first recovered water”.

After the first rinsing step, the first recovered water was monitored and analyzed. The content of peroxides within the first recovered water is in the range of 200-1500 ppm.

It was noticed that the concentration of peroxides in the first recovered water was compatible for being mixed with the sterilizing solution of the first tank 11 without affecting it.

Thus, the first recovered water is used for sterilizing new containers 100 in the sterilizing unit 10.

After the first rinsing step, the process comprises a second rinsing step which is carried out in a second rinsing tract P2 of the path P.

For the second rinsing step it is employed a second amount of sterile water coming from the second tank 21.

The second amount of sterile water is dispensed towards the containers 100 when they are moving along the second rinsing tract P2.

In particular, the second amount of sterile water is dispensed by nozzles (not illustrated) which are in selective fluid communication with the UHT device 22 (and therefore with the second tank 21).

In one embodiment of the process, the dispensing means 23 (i.e. the nozzles) are configured to dispense the sterile water by a single dispensing pulse (without any interruption) that spans both the first rinsing step and the second rinsing step. In other words, there is no interruption between dispensing the first amount of sterile water and the second amount of sterile water.

In another embodiment of the process, the dispensing means 23 are configured to dispense the first amount of sterile water with a first dispensing pulse, and the second amount of sterile water with a second dispensing pulse. In practice, the second dispensing pulse occurs after the first dispensing pulse has finished.

At least a part of exhaust sterile water used during the second rinsing step is recovered for being used in rinsing new containers 100.

In particular, part of the exhaust sterile water dropping from the containers 100 which undergo the second rinsing step is collected by the recovery collector 24 and sent back to the UHT device 22.

This part of exhaust sterile water recovered from the second rinsing step is called here “second recovered water”.

After the second rinsing step, the second recovered water was monitored and analyzed. The content of peroxides within the second recovered water is in the range of 10-100 ppm.

This concentration of peroxides is lower than the concentration of peroxides in the first recovered water. This is due to the fact that containers 100 undergoing the second rinsing have already been rinsed in the first rinsing step, thus they have lower peroxide residues.

In particular, it was noticed that the concentration of peroxides in the second recovered water was sufficiently low for being compatible with the UHT device 22 for generating sterile water.

Thus, the second recovered water is used for rinsing new containers 100 in the rinsing unit 20, in particular in the first rinsing tract P1 and/or in the second rinsing tract P2.

As said, only part of the exhaust sterile water used to rinse the containers 100 is recovered and may be used in the next rinsing cycles.

There is a part of exhaust sterile water which drops from the containers 100 and is neither collected by the recovery collector 24 nor by the further recovery collector 14. This part of exhaust sterile water may be discharged by means of a discharge outlet or may be channelized and reused in another way within the apparatus 1.

The characteristics of the apparatus and process for sterilizing and rinsing containers, according to the present invention, are clear, as are the advantages.

In particular, the invention allows a partial recovery of the exhaust sterile water used for rinsing the containers. Depending on the content of peroxides the recovered exhaust sterile water may be used for sterilizing or for rinsing new containers.

In particular, after the second rinsing step the content of peroxides is sufficiently lower for allowing the recovered exhaust sterile water to be used for next rinsing after having been thermally treated. There is no need to further treat or filter this recovered water since the content of peroxides is sufficiently low to be submitted as is to the UHT device.

The recovery of part of the sterile water for next rinsing steps is even more advantageous since the consumption of fresh water to produce sterile water is reduced.

1. An apparatus (1) for sterilizing and rinsing containers (100), comprising:
a sterilizing unit (10) configured to treat the containers (100) by means of a sterilizing solution contained in a first tank (11);
a rinsing unit (20) arranged downstream the sterilizing unit (10) and configured to rinse the containers (100) advancing along a path (P) with sterile water;
a UHT device (22) configured to provide sterile water to the rinsing unit (20);
dispensing means (23) in selective fluid communication with the UHT device (22) so as to dispense a first amount of sterile water towards the containers (100) in a first rinsing tract (P1) of the path (P) and a second amount of sterile water towards the containers (100) in a second rinsing tract (P2) of the path (P) that is downstream the first rinsing tract (P1);
a recovery collector (24) arranged at the second rinsing tract (P2) of the path (P) for collecting at least part of exhaust sterile water dropping from the containers (100) advancing along the second rinsing tract (P2), said recovery collector (24) being in selective fluid communication with the UHT device (22).

2. The apparatus (1) according to claim 1, comprising a further recovery collector (14) arranged at the first rinsing tract (P1) of the path (P) for collecting at least part of the exhaust sterile water dropping from the containers (100) advancing along the first rinsing tract (P1), said further recovery collector (14) being in selective fluid communication with the first tank (11).

3. The apparatus (1) according to claim 2, wherein the recovery collector (24) and the further recovery collector (14) are separated.

4. The apparatus (1) according to claim 3, comprising two distinct piping systems (25, 15) configured to establish a selective fluid communication respectively between the recovery collector (24) and the UHT device (22) and between the further recovery collector (14) and the first tank (11).

5. The apparatus (1) according to claim 1, wherein the sterilizing unit (10) and the rinsing unit (20) comprise a corresponding rotating carousel.

6. The apparatus (1) according to claim 1, wherein the sterilizing unit (10) and the rinsing unit (20) comprise a corresponding linear conveyor or belt.

7. The apparatus (1) according to claim 1, further comprising a second tank (21) arranged downstream the UHT device (22) so as to receive the sterile water from it, said second tank (21) being configured to supply the sterile water to the dispensing means (23).

8. The apparatus (1) according to claim 1, wherein said dispensing means (23) are configured to dispense sterile water by a single dispensing pulse which supplies both the first amount of sterile water and the second amount of sterile water.

9. A process for sterilizing and rinsing containers (100), the process comprising the following steps:
sterilizing the containers (100) with a sterilizing solution coming from a first tank (11);
after sterilization, subjecting the containers (100) to a first rinsing by dispensing towards them a first amount of sterile water from a UHT device (22) in a first rinsing tract (P1) of an advancement path (P) of the containers (100);
after the first rinsing, subjecting the containers (100) to a second rinsing by dispensing towards them a second amount of sterile water from said UHT device (22) in a second rinsing tract (P2) of the path (P);
collecting at least part of exhaust sterile water dropping from the containers (100) undergoing the second rinsing and sending it to the UHT device (22).

10. The process of claim 9, wherein the exhaust sterile water which is sent to the UHT device (22) is used in rinsing new containers (100) advancing along the path (P).

11. The process of claim 9, wherein the first rinsing step and the second rinsing step are performed by dispensing means (23) which are configured to dispense sterile water by a single dispensing pulse which spans both the first rinsing step and the second rinsing step.

12. The process of claim 11, wherein the dispensing means (23) are configured to dispense the first amount of sterile water and the second amount of sterile water without any interruption.

13. The process of claim 8, wherein the sterile water obtained in the UHT device (22) is sent to a second tank (21) which in turn is configured to dispense the sterile water towards the containers (100) during the first rising and the second rinsing.

14. The process of claim 8, further comprising a step of collecting at least part of exhaust sterile water dropping from the containers (100) undergoing the first rinsing and sending it to the first tank (11).

15. The process according to claim 14, wherein the exhaust sterile water collected in the first rinsing tract (P1) is mixed with the sterilizing solution of the first tank (11) for being used in sterilizing new containers (100).
